SEQUENCE LISTING (6 sequences)

SEQ ID NO: 1 - DNA, 31 bp, Artificial Sequence
  Synthetic DNA
gaattcatggacaaaccgcgtcacgaacgtc

SEQ ID NO: 2 - DNA, 31 bp, Artificial Sequence
  Synthetic DNA
aagctttcacagtatttggctgagatggagc

SEQ ID NO: 3 - DNA, 31 bp, Artificial Sequence
  Synthetic DNA
gaattcatgaataatgtagccgctaaaaatg

SEQ ID NO: 4 - DNA, 27 bp, Artificial Sequence
  Synthetic DNA
ctgcagtcaagattgcttagaggcttc

SEQ ID NO: 5 - DNA, 30 bp, Artificial Sequence
  Synthetic DNA
tcatgagatctcagtgcaatttatctcttc

SEQ ID NO: 6 - DNA, 34 bp, Artificial Sequence
  Synthetic DNA
tcatgagatctgccaagggttggtttgcgcattc

(19) United States
(12) Patent Application Publication
(10) Pub. No.: US 2013/0330794 A1
(43) Pub. Date: Dec. 12, 2013

(54) METHOD FOR PRODUCING 2,3-BUTANEDIOL BY FERMENTATION

(71) Applicant: Rupert Pfaller, Muenchen (DE)

(72) Inventor: Rupert Pfaller, Muenchen (DE)

(73) Assignee: WACKER CHEMIE AG, Muenchen (DE)

(21) Appl. No.: 13/979,878

(22) PCT Filed: Jan. 30, 2012
(86) PCT No.: PCT/EP2012/051426

§ 371 Date: Jul. 16, 2013

(30) Foreign Application Priority Data
Jan. 31, 2011 (DE) 10 2011 003 394.7

(51) Int. Cl. C12P 7/18; C12N 15/74

(52) U.S. Cl. CPC C12P 7/18; C12N 15/74
USPC 435/158; 435/252.1; 435/252.3

(57) ABSTRACT

The invention relates to a production strain for producing 2,3-butanediol. Said production strain has an acetolactate synthase activity 2—68 times higher than the original strain. The invention also relates to a method for producing 2,3-butanediol by fermentation by means of said production strain.

The invention relates to a method for producing 2,3-butanediol (2,3-BDL) by fermentation by means of an improved microorganism strain which has an acetolactate synthase activity increased 2 to 68 times compared to the unimproved original strain.

Because of increasing crude oil prices, the production costs of the base materials for the chemical industry obtained from them by petrochemistry are also increasing. This results in a growing interest in alternative production methods for chemical base materials, especially on the basis of renewable raw materials.

Examples of chemical base materials (so-called chemical synthesis building blocks) from renewable raw materials are ethanol (C2 building block), glycerine, 1,3-propanediol, 1,2-propanediol (C3 building blocks) or succinic acid, 1-butanol, 2-butanol, 1,4-butanediol or also 2,3-butanediol (C4 building blocks). These chemical building blocks are the biogenic starting compounds from which further base chemicals can then be produced by chemical routes. The prerequisite for this is the inexpensive production of the particular synthesis building blocks from renewable raw materials by fermentation. Decisive cost factors are on the one hand the availability of suitable cheap renewable raw materials and on the other hand efficient microbial fermentation methods which efficiently convert these raw materials into the desired chemical base material. Here it is decisive that the microorganisms used produce the desired product in high concentration and with little by-product formation from the biogenic raw material. The optimization of the microorganisms designated as production strains with regard to productivity and profitability is for example achieved by metabolic engineering.

A C4 building block accessible by a fermentative route is 2,3-butanediol. The prior art on the production of 2,3-butanediol by fermentation is summarized in Celinska and Grajek (Biotechnol. Advances (2009) 27: 715-725). 2,3-butanediol is a possible starting product for products of petrochemistry with four C atoms (C4 building blocks) such as acetoin, diacetyl, 1,3-butadiene and 2-butanone (methyl ethyl ketone, MEK). In addition, products with two C atoms (C2 building blocks) such as acetic acid (DE 102010001399) and, derived therefrom, acetaldehyde, ethanol and even ethylene are accessible. Through dimerization of 2,3-butanediol, C8 compounds, which are used for example as fuel in the air travel sector, are also feasible.

The biosynthetic pathway to 2,3-butanediol trodden by various microorganisms is known (see review by Celinska and Grajek, Biotechnol. Advances (2009) 27: 715-725) and leads from the central metabolic product pyruvate via the three following enzymatic steps to 2,3-butanediol:

Reaction of acetolactate synthase: formation of acetolactate from two molecules of pyruvate with elimination of CO₂.

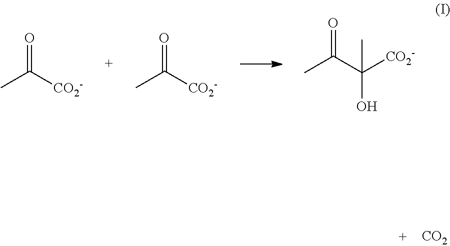

Reaction of acetolactate decarboxylase: decarboxylation of acetolactate to acetoin.

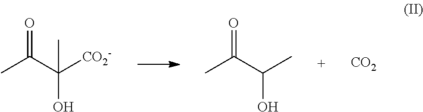

Reaction of acetoin reductase (2,3-butanediol dehydrogenase): NADH-dependent reduction of acetoin to 2,3-butanediol.

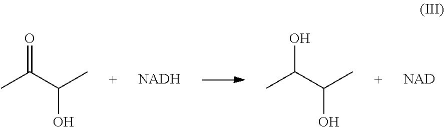

Various natural producers of 2,3-butanediol are known, e.g. from the genera Klebsiella, Raoultella, Enterobacter, Aerobacter, Aeromonas, Serratia, Bacillus, Paenibacillus, Lactobacillus, Lactococcus etc. But yeasts are also known as producers (e.g. baker's yeast). Most bacterial producers are microorganisms of biosafety level S2, and can thus not be used on an industrial scale without laborious and costly industrial safety measures (concerning this and other microbial 2,3-butanediol producers see review by Celinska and Grajek, Biotechnol. Advances (2009) 27: 715-725). This would also apply for not previously described genetically optimized production strains based on these strains.

Production strains of the biosafety level S1 are preferable for cost-efficient industrial scale 2,3-BDL production. However, sufficiently high 2,3-BDL yields have not previously been described for these. Known 2,3-BDL production strains from the biosafety level S1 are strains of the species Klebsiella terrigena, Klebsiella planticola and strains of the genus Bacillus (or Paenibacillus) such as Bacillus polymyxa or Bacillus licheniformis. In the technical literature (Drancourt et al., Int. J. Syst. Evol. Microbiol. (2001), 51: 925-932) as a result of a change in taxonomic nomenclature, the species Klebsiella terrigena and Klebsiella planticola are also synonymously designated as Raoultella terrigena and Raoultella planticola. These are strains of the same species.

For these strains classified in safety level S1, 2,3-butanediol yields of not more than 57 g/l (637 mM, production time 60 hrs) have previously been reported (Nakashimada et al., J. Bioscience and Bioengineering (2000) 90: 661-664). These yields are far too low for profitable production. >80 g/1 2,3-butane-diol (fermentation time maximum 72 hrs), preferably >100 g/l 2,3-butanediol, are regarded as the minimum fermentation yield for profitable production.

A current definition for the term “biosafety level” and the classification into various safety levels can for example be found in “Biosafety in Microbiological and Biomedical Laboratories”, p. 9ff. (Table 1), Centers for Disease Control and Prevention (U.S.) (Editor), Public Health Service (U.S.) (Editor), National Institutes of Health (Editor), Publisher: U.S. Dept. of Health and Human Services; 5, 5th Edition, Revised December 2009 edition (Mar. 15, 2010); ISBN-10: 0160850428.

The overexpression of acetolactate synthase in the lactic acid fermenting bacterium Lactococcus lactis is described in Platteeuw et al., Appl. Environ. Microbiol. (1995) 61: 3967-3971. There the utilization of the C source lactose and the product spectrum resulting therefrom were studied. The purpose of the studies was optimization of the production of the aroma substance diacetyl. In strains which overexpressed acetolactate synthase 100-fold, no 2,3-butanediol could be detected. In a strain overexpressing acetolactate synthase 100-fold and lactose dehydrogenase deficient, yields of at most 22.3 mM (corresponds to 2 g/l) 2,3-butanediol could be achieved. These yields lie far below the yields achievable in S1 wild type strains. In addition, these results were only achieved with the use of the milk sugar lactose obtained from animal sources, which is too expensive for industrial scale use.

The purpose of the invention was to provide production strains for producing 2,3-butanediol which enable markedly higher 2,3-butanediol yields than the original strain.

The problem was solved by means of a production strain which can be produced from an original strain, characterized in that the production strain has an acetolactate synthase activity lying 2 to 68 times higher than the original strain.

In the present invention, a distinction is made between an original strain and a production strain. The original strain can be a wild type strain which is not further optimized, but is capable of 2,3-butanediol production, or a wild type strain which is already further optimized. However, in a wild type strain which is already further optimized (e.g. effected through a genetic engineering operation) the acetolactate synthase activity is not affected by the optimization.

In the sense of the present invention, a production strain should be understood to mean an original strain optimized as regards 2,3-butanediol production, which is characterized by increased activity of the enzyme acetolactate synthase (ALS) in comparison to the original strain. The production strain is produced from the original strain. If an already optimized original strain is to be further improved by an increase in the acetolactate synthase activity, then it is naturally also possible first to increase the acetolactate synthase activity in an unimproved strain and then to introduce further improvements.

Here the increase in the acetolactate synthase activity in the production strain can be caused by any mutation in the genome of the original strain (e.g. a mutation increasing the promoter activity), a mutation in the acetolactate synthase gene increasing the enzyme activity or by overexpression of a homologous or else also heterologous acetolactate synthase gene in the original strain.

Preferably the overexpression is of a homologous or of a heterologous acetolactate synthase gene in the original strain.

Preferably, in the production strain the acetolactate synthase activity is increased by the factor 3 to 64, particularly preferably by the factor 3 to 30, and especially preferably by the factor 4 to 20 compared to the original strain.

Particularly preferably, this increased acetolactate synthase activity is achieved by an increased expression of a homologous or heterologous gene coding for an acetolactate synthase enzyme compared to the original strain.

The original strain can be any 2,3-butanediol producing strain. Preferably it is a strain of the genus Klebsiella, Raoultella, Bacillus or Lactobacillus.

Particularly preferably it is a strain of the species Klebsiella (Raoultella) terrigena, Klebsiella (Raoultella) planticola, Bacillus (Paenibacillus) polymyxa or Bacillus licheniformis, wherein a strain of the species Klebsiella (Raoultella) terrigena or Klebsiella (Raoultella) planticola is even more preferable.

Particularly preferably, it is an original strain and a production strain classified in the safety level S1 and among these, more preferably, strains of the species Klebsiella (Raoultella) terrigena or Klebsiella (Raoultella) planticola.

Since in the acetolactate synthase overexpressing Lactococcus strain known from Platteeuw et al., Appl. Environ. Microbiol. (1995) 61: 3967-3971, no 2,3-butanediol could be detected, those skilled in the art assumed that the overexpression of acetolactate synthase cannot cause any improvement in 2,3-butanediol production in a production strain. In the context of the experiments which led to the present invention, it was surprisingly found that through a limited (3.2 to 68.4 fold) increased ALS activity in a production strain, the yield of 2,3-butanediol can be significantly increased. Preferably, the increased ALS activity is achieved by recombinant overexpression of an acetolactate synthase (EC 2.2.1.6).

As shown in the examples of the present application, an over-expression of the acetolactate synthase by the factor 3.2 to up to a factor of 68.4 (see example 3) is suitable for increasing the 2,3-butanediol yield (determined as 2,3-butanediol volume yield in g/l) in shake flasks by more than 25%, preferably more than 30%, and in particular more than 40% (see example 4) and in the fermentation by more than 15%, preferably more than 20% and especially preferably by more than 30% (see examples 5 and 6).

As further shown in examples 3 and 4, the recombinant overexpression of acetolactate synthase by a factor of 68 enables an increase in the 2,3-butanediol yield in the shake flask.

Acetolactate synthase (ALS) is an enzyme from the enzyme class EC 2.2.1.6. It can be any gene-coded enzyme which causes the synthesis of acetolactate from two molecules of pyruvate according to formula (I).

In a preferable embodiment, the gene of the acetolactate synthase derives from a bacterium of the genus Klebsiella (Raoultella) or Bacillus.

In a particularly preferable embodiment, the gene of the acetolactate synthase derives from a strain of the species Klebsiella terrigena, Klebsiella planticola, Bacillus (Paenibacillus) polymyxa or Bacillus licheniformis and in particular from a strain of the species Klebsiella terrigena, Klebsiella planticola or Bacillus licheniformis. These strains are all commercially available e.g. from the DSMZ Deutsche Sammlung von Mikroorganismen and Zellkulturen GmbH (Braunschweig), and among these the strain Klebsiella (Raoultella) terrigena DSM 2687 used in the examples is also commercially available from DSMZ GmbH.

The strain according to the invention thus makes it possible to increase the production of acetolactate by fermentation. The invention thus enables not only the production of 2,3-butane-diol but also the production of other metabolic products which like 2,3-butanediol can be derived from acetolactate. These metabolic products include acetoin, diacetyl, ethanol and acetic acid.

In a preferable embodiment, a production strain according to the invention is also characterized in that it was produced from an original strain, as defined in the application, and produces an acetolactate synthase in recombinant form with the result that its 2,3-BDL production (volume production expressed in g/l 2,3-BDL) is increased compared to the non-genetically optimized original strain by at least 25%, preferably 50%, particularly preferably 75% and especially preferably by 100%, wherein the 2,3-butanediol yield of the original strain is at least 90 g/l.

The production strain according to the invention is preferably produced by introduction of a gene construct into one of the said original strains.

The gene construct in its simplest form is defined as consisting of the acetolactate synthase structural gene, operatively linked to which a promoter is positioned upstream. Optionally, the gene construct can also comprise a terminator which is positioned downstream from the acetolactate synthase structural gene. A strong promoter which leads to strong transcription is preferred. Preferable among the strong promoters is the so-called “Tac promoter” familiar to those skilled in the art from the molecular biology of E. coli.

The gene construct can in a manner known per se be present in the form of an autonomously replicating plasmid, wherein the copy number of the plasmid can vary. A large number of plasmids which depending on their genetic structure can replicate in autonomous form in a given production strain are known to those skilled in the art.

The gene construct can however also be integrated in the genome of the production strain, wherein any gene location along the genome is suitable as an integration site.

The gene construct, either in plasmid form or with the purpose of genomic integration, is introduced into the production strain in a manner known per se by genetic transformation. Various methods of genetic transformation are known to those skilled in the art (Aune and Aachmann, Appl. Microbiol. Biotechnol. (2010) 85: 1301-1313), including for example electroporation.

For the selection of transformed production strains, the gene construct, also in a known manner, contains a so-called selection marker for the selection of transformants with the desired gene construct. Known selection markers are selected from antibiotic resistance markers or from the selection markers complementing an auxotrophy.

Antibiotic resistance markers, particularly preferably those which impart resistance towards antibiotics selected from ampicillin, tetracycline, kanamycin, chloramphenicol or zeocin, are preferable.

In a preferable embodiment, a production strain according to the invention thus contains the gene construct, either in plasmid form or integrated into the genome, and produces an acetolactate synthase enzyme in recombinant form. The recombinant acetolactate synthase enzyme is capable of producing acetolactate from two molecules of pyruvate with elimination of CO₂. Now it has surprisingly been found that by suitable recombinant overexpression of the acetolactate synthase enzyme in the production strain the 2,3-butanediol yield can be significantly increased compared to the original strain.

The original strain can be a not further optimized wild type strain. The original strain can however already have been previously optimized, and be further optimized as an acetolactate synthase producing production strain according to the invention. The optimization of an acetolactate synthase producing production strain according to the invention comprised by the invention can on the one hand be effected by mutagenesis and selection of mutants with improved production properties. The optimization can however also be effected genetically by additional expression of one or more genes which are suitable for improving the production properties. Examples of such genes are the already mentioned 2,3-butanediol biosynthesis genes acetolactate decarboxylase and acetoin reductase. These genes can be expressed in the production strain in a manner known per se each as individual gene constructs or else also combined as one expression unit (as a so-called operon). Thus for example it is known that in Klebsiella terrigena all three biosynthesis genes for 2,3-butanediol (so-called BUD operon, Blomqvist et al., J. Bacteriol. (1993) 175: 1392-1404), and in strains of the genus Bacillus the genes of acetolactate synthase and acetolactate decarboxylase, are organised in an operon (Renna et al., J. Bacteriol (1993) 175: 3863-3875).

Furthermore, the production strain can be optimized by inactivating one or more genes whose gene products have an adverse effect on the 2,3-butanediol production. Examples of these are genes whose gene products are responsible for byproduct formation. These include for example lactate dehydrogenase (lactic acid formation), acetaldehyde dehydrogenase (ethanol formation) or else also phosphotransacetylase, or acetate kinase (acetate formation).

Furthermore, the invention comprises a method for producing 2,3-butanediol by means of a production strain according to the invention.

The method is characterized in that cells of a production strain according to the invention are cultured in a growth medium. During this, on the one hand biomass of the production strain and on the other hand the product 2,3-BDL are formed. The formation of biomass and 2,3-BDL during this correlates in time or else take place decoupled in time from one another. The culturing is effected in a manner familiar to those skilled in the art. For this, the growth can be effected in shake flasks (laboratory scale) or else also by fermentation (production scale).

A method on the production scale by fermentation is preferable, wherein as the production scale a fermentation volume greater than 10 l is particularly preferable and a fermentation volume greater than 300 l is especially preferable.

Growth media are familiar to those skilled in the art from the practice of microbial culturing. They typically consist of a carbon source (C source), a nitrogen source (N source) and additives such as vitamins, salts and trace elements through which the cell growth and the 2,3-BDL product formation are optimized. C sources are those which can be utilized by the production strain for forming the 2,3-BDL product. These include all forms of monosaccharides, comprising C6 sugars such as for example glucose, mannose or fructose and C5 sugars such as for example xylose, arabinose, ribose or galactose.

However, the production method according to the invention also comprises all C sources in the form of disaccharides, in particular saccharose, lactose, maltose or cellobiose.

The production method according to the invention further also comprises all C sources in the form of higher saccharides, glycosides or carbohydrates with more than two sugar units such as for example maltodextrin, starch, cellulose, hemicellulose, pectin and monomers or oligomers liberated therefrom by hydrolysis (enzymatic or chemical). Here the hydrolysis of the higher C sources can be positioned upstream of the production method according to the invention or else be effected in situ during the production method according to the invention.

Other utilizable C sources different from sugars or carbohydrates are acetic acid (or acetate salts derived therefrom), ethanol, glycerine, citric acid (and salts thereof) or pyruvate (and salts thereof). However, gaseous C sources such as carbon dioxide or carbon monoxide are also feasible.

The C sources concerned in the production method according to the invention comprise both the isolated pure substances but also, to increase the profitability, not further purified mixtures of the individual C sources, as they can be obtained as hydrolysates by chemical or enzymatic digestion of the plant raw materials. These for example include hydrolysates of starch (monosaccharide glucose), of sugar beet (monosaccharides glucose, fructose and arabinose), of sugar cane (disaccharide saccharose), of pectin (monosaccharide galacturonic acid) or also of lignocellulose (monosaccharide glucose from cellulose, monosaccharides xylose, arabinose, mannose and galactose from hemicellulose and lignin, not a member of the carbohydrates). Furthermore, waste products from the digestion of plant raw materials, such as for example molasses (sugar beet) or bagasse (sugar cane) can also be used as C sources.

Preferable C sources for culturing the production strains are glucose, fructose, saccharose, mannose, xylose, arabinose and plant hydrolysates which can be obtained from starch, ligno-cellulose, sugar cane or sugar beet.

A particularly preferable C source is glucose, either in isolated form or as a component of a plant hydrolysate.

N sources are those which can be utilized by the production strain for the formation of biomass. These include ammonia, gaseous or in aqueous solution as NH₄OH or else also salts thereof such as for example ammonium sulfate, ammonium chloride, ammonium phosphate, ammonium acetate or ammonium nitrate. Also suitable as an N source are the known nitrate salts such as for example KNO₃, NaNO₃, ammonium nitrate, Ca(NO₃)₂, Mg(NO₃)₂ and other N sources such as for example urea. The N sources also include complex amino acid mixtures such as for example yeast extract, proteose peptone, malt extract, soya peptone, casamino acids, corn steep liquor (liquid or else also dried as so-called CSD) and also NZ-Amine and yeast nitrogen base.

The culturing can be effected in so-called batch mode, wherein the growth medium is inoculated with a starter culture of the production strain and then the cell growth takes place with no further feeding of nutrient sources.

The culturing can also be effected in the so-called fed batch mode, wherein after an initial phase of growth in batch mode, additional nutrient sources are fed in (Feed) in order to compensate for their consumption. The feed can consist of the C source, the N source, one or more vitamins important for production, or trace elements or of a combination of the aforesaid. Here, the feed components can be metered in together as a mixture or else also separately in individual feed periods. In addition, other medium components and additives specifically increasing 2,3-BDL production can also be added to the feed. In this case, the feed can be introduced continuously or in portions (discontinuously) or else also in a combination of continuous and discontinuous feed. Culturing according to the fed batch mode is preferable.

Preferable C sources in the feed are glucose, saccharose, molasses, or plant hydrolysates which can be obtained from starch, lignocellulose, sugar cane or sugar beet.

Preferable N sources in the feed are ammonia, gaseous or in aqueous solution as NH₄OH and its salts ammonium sulfate, ammonium phosphate, ammonium acetate and ammonium chloride, and furthermore urea, KNO₃, NaNO₃ and ammonium nitrate, yeast extract, proteose peptone, malt extract, soya peptone, casamino acids, corn steep liquor and also NZ-Amine and yeast nitrogen base.

Particularly preferable N sources in the feed are ammonia, or ammonium salts, urea, yeast extract, soya peptone, malt extract or corn steep liquor (liquid or in dried form).

The culturing is effected under pH and temperature conditions which favor the growth and the 2,3-BDL production of the production strain. The utilizable pH range extends from pH 5 to pH 8. A pH range from pH 5.5 to pH 7.5 is preferable. A pH range from pH 6.0 to pH 7 is particularly preferable.

The preferable temperature range for the growth of the production strain is 20° C. to 40° C. The temperature range from 25° C. to 35° C. is particularly preferable.

The growth of the production strain can be effected facultatively without oxygen input (anaerobic culturing) or else also with oxygen input (aerobic culturing). Aerobic culturing with oxygen, wherein the oxygen supply is ensured by introduction of compressed air or pure oxygen, is preferable. Aerobic culturing by introduction of compressed air is particularly preferable.

The culturing time for 2,3-BDL production is between 10 hrs and 200 hrs. A culturing time of 20 hrs to 120 hrs is preferable. A culturing time of 30 hrs to 100 hrs is particularly preferable.

Culture mixtures which are obtained by the method described above contain the 2,3-BDL product, preferably in the culture supernatant. The 2,3-BDL product contained in the culture mixtures can either be further used directly without further processing or else can be isolated from the culture mixture. For the isolation of the 2,3-BDL product, process steps known per se are available, including centrifugation, decantation, filtration, extraction, distillation or crystallization, or precipitation. These process steps can however be combined in any desired form in order to isolate the 2,3-BDL product in the desired purity. The degree of purity to be attained thereby is dependent on the subsequent use of the 2,3-BDL product.

Various analytical methods are available for identification, quantification and determination of the degree of purity of the 2,3-BDL product, including NMR, gas chromatography, HPLC, mass spectroscopy or also a combination of these analytical methods.

The figures show the plasmids mentioned in the examples.

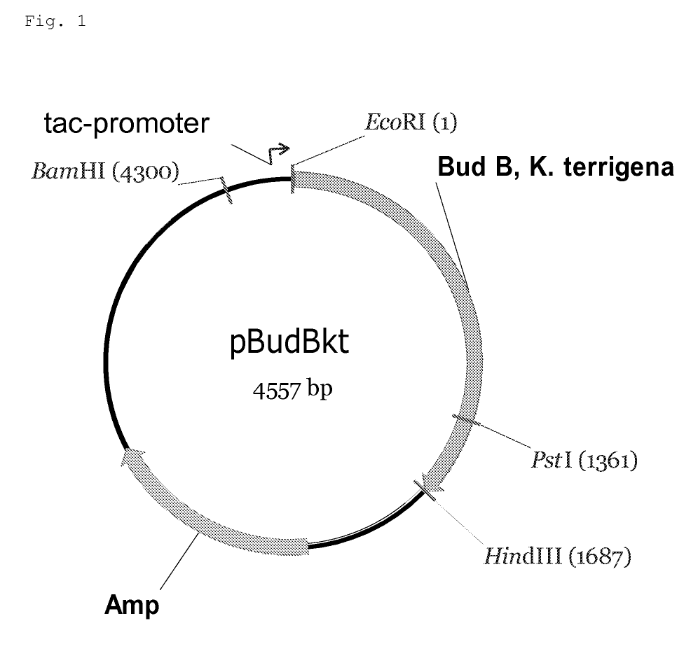

FIG. 1 shows the 4.9 kb sized acetolactate synthase expression vector pBudBkt produced in example 1.

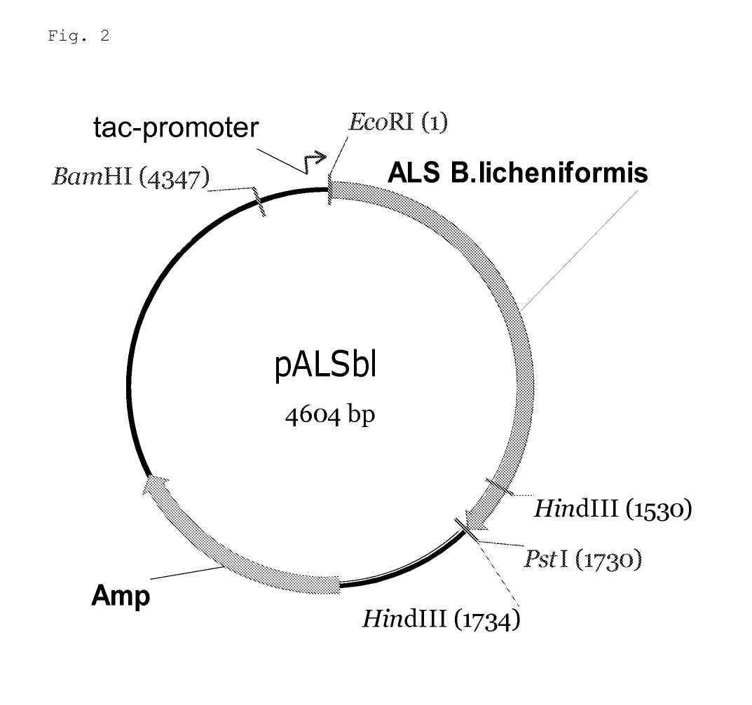

FIG. 2 shows the 4.9 kb sized acetolactate synthase expression vector pALSbl produced in example 1.

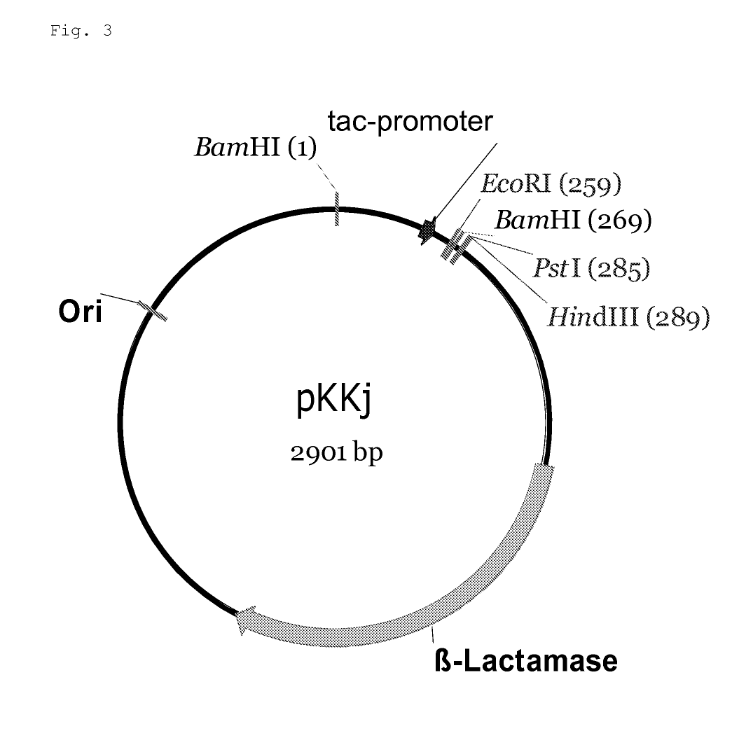

FIG. 3 shows the 2.9 kb sized expression vector pKKj produced in example 1.

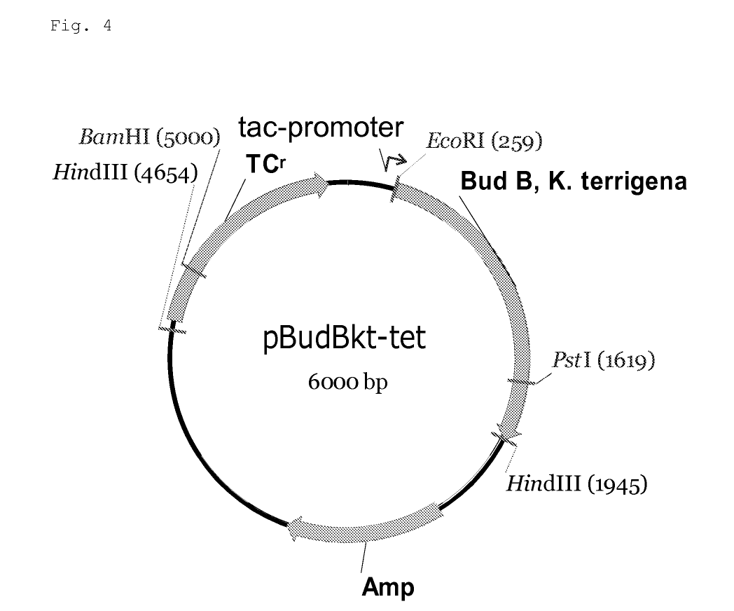

FIG. 4 shows the 6 kb sized expression vector pBudBkt-tet produced in example 1.

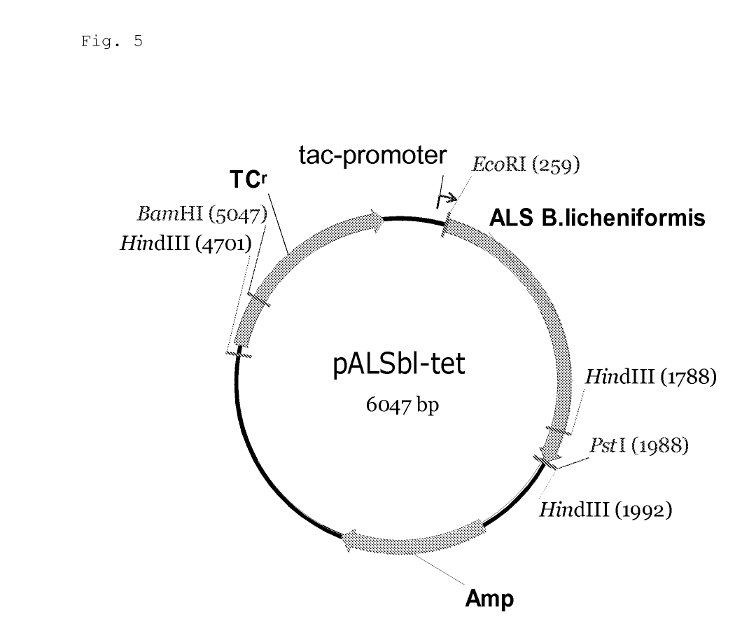

FIG. 5 shows the 6 kb sized expression vector pALSbl-tet produced in example 1.

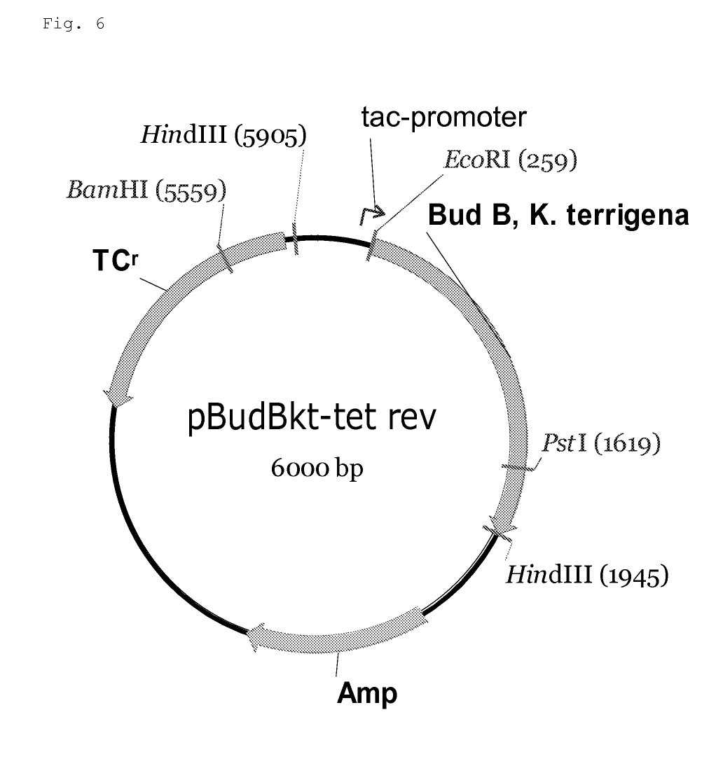

FIG. 6 shows the 6 kb sized plasmid pBudBkt-tet(rev) used in example 1.

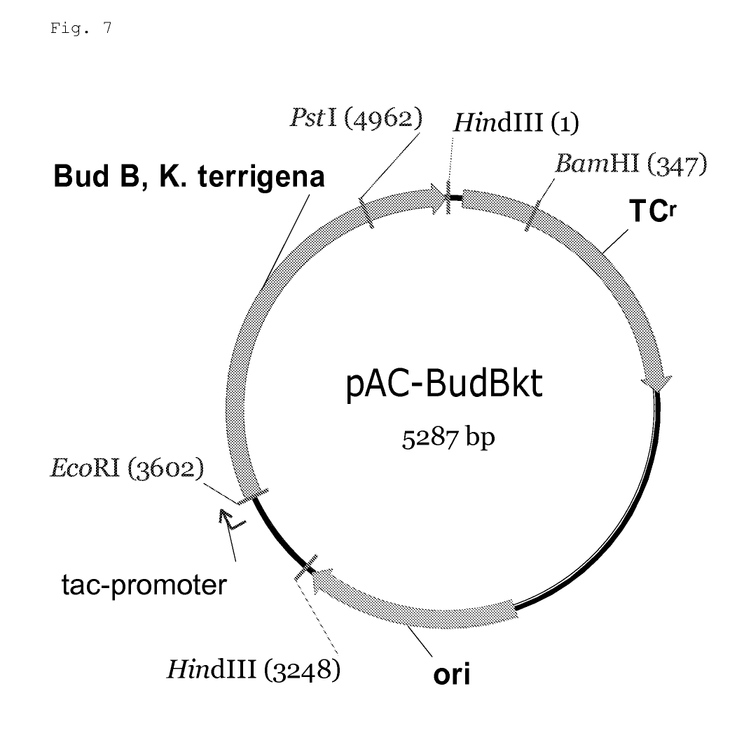

FIG. 7 shows the 5.3 kb sized expression vector pAC-BudBkt produced in example 1.

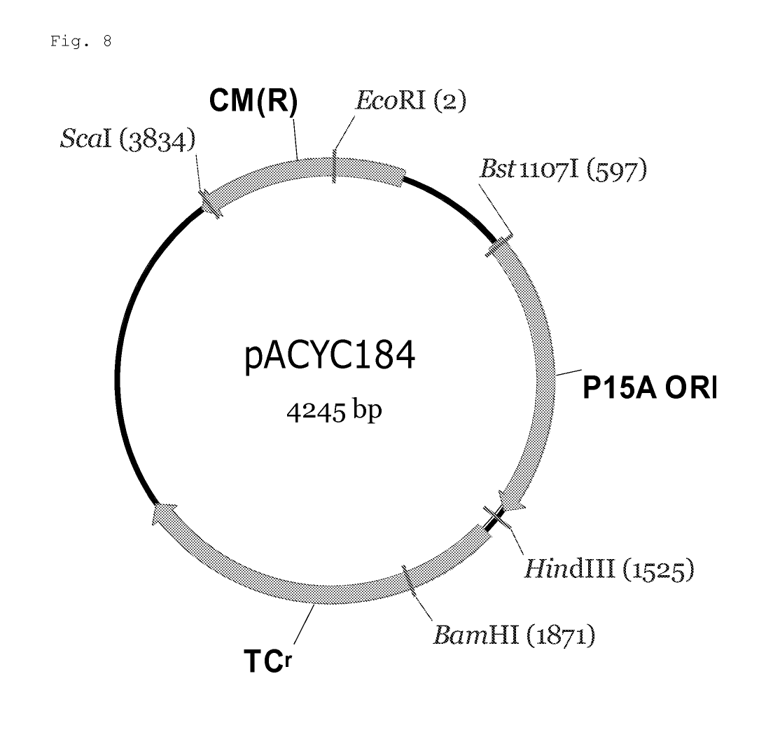

FIG. 8 shows the plasmid pACYC184 used in example 1.

The invention is further explained by the following examples:

### EXAMPLE 1

Production of Acetolactate Synthase Expression Vectors

The acetolactate synthase genes from K. terrigena and B. licheniformis were used. The DNA sequence of the acetolactate synthase gene from K. terrigena is disclosed in the “GenBank” gene database under the access number L04507, by 969-2648. It was isolated as a DNA fragment of 1.7 kb size in a PCR reaction (Taq DNA polymerase, Qiagen) from genomic K. terrigena DNA (strain DSM 2687, commercially available from the DSMZ Deutsche Sammlung von Mikroorganismen and Zellkulturen GmbH) with the primers BUD5f and BUD6r.

The DNA sequence of the acetolactate synthase gene from B. licheniformis is disclosed in the “GenBank” gene database under the access number NC_{—}006270, under which the whole genome sequence of B. licheniformis is disclosed. The acetolactate synthase gene can be found there in complementary form from by 3675290-3677008. It was isolated as a DNA fragment of 1.7 kb size in a PCR reaction (Taq DNA polymerase, Qiagen) from genomic B. licheniformis DNA (strain DSM 13, commercially available from DSMZ GmbH) with the primers BLals-1f and BLals-2r.

The genomic DNA used for the PCR reactions had previously been obtained in a manner known per se with a DNA isolation kit (Qiagen) from cells from the culturing of K. terrigena DSM 2687 and B. licheniformis DSM 13 in LB medium (10 g/l tryptone, 5 g/l yeast extract, 5 g/l NaCl).

Primers BUD5f (SEQ ID NO: 1) and BUD6r (SEQ ID NO: 2) had the following DNA sequences:

SEQ ID NO: 1:
5′-GAA TTC ATG GAC AAA CCG CGT CAC GAA CGT C-3′
SEQ ID NO: 2:
5′-AAG CTT TCA CAG TAT TTG GCT GAG ATG GAG C-3′

Primers BLals-1f (SEQ ID NO: 3) and BLals-2r (SEQ ID NO: 4) had the following DNA sequences:

SEQ ID NO: 3:
5′-GAA TTC ATG AAT AAT GTA GCC GCT AAA AAT G-3′
SEQ ID NO: 4:
5′-CTG CAG TCA AGA TTG CTT AGA GGC TTC-3′

The PCR products were then digested with Eco RI (contained in primers BUD5f and BLals-1f) and Hind III (contained in primer BUD6r) and Pst I (contained in primer BLals-2r) respectively and cloned into the expression vector pKKj. In this way, the acetolactate synthase expression vectors pBudBkt (FIG. 1) and pALSbl (FIG. 2) each 4.9 kb in size, were formed.

Expression Vector pKKj:

pKKj is a derivative of the expression vector pKK223-3. The DNA sequence of pKK223-3 is disclosed in the “GenBank” gene data-base under the access number M77749.1. From the 4.6 kb plasmid ca. 1.7 kb were removed (bp 262-1947 of the DNA sequence disclosed in M77749.1), as a result of which the 2.9 kb expression vector pKKj (FIG. 3) was formed.

The expression vectors pBudBkt and pALSbl were modified by incorporation of an expression cassette for the tetracycline resistance gene. For this, the tetracycline resistance gene was first isolated from the plasmid pACYC184 (the DNA sequence of pACYC184 is accessible in the “Genbank” gene database under the access number X06403.1, see also FIG. 8) by PCR (Taq DNA polymerase, Qiagen) with the primers tet1f and tet2r and subsequent digestion with Bgl II (cleavage sites contained in the primers tet1f and tet2r) as a 1.45 kb fragment and then cloned into the vectors pBudBkt and pALSbl each cleaved with Bam HI. As a result, the expression vectors pBudBkt-tet (FIG. 4) and pALSbl-tet (FIG. 5), each 6 kb in size, were formed. As shown in FIG. 4 and FIG. 5, in each case a clone was selected in which the tetracycline and the acetolactate synthase expression cassettes were each oriented in the same direction.

Primers tet1f (SEQ ID NO: 5) and tet2r (SEQ ID NO: 6) had the following DNA sequences:

SEQ ID NO: 5:
5′-TCA TGA GAT CTC AGT GCA ATT TAT CTC TTC-3′
SEQ ID NO: 6:
5′-TCA TGA GAT CTG CCA AGG GTT GGT TTG CGC ATT
C-3′

Expression Vector pAC-BudBkt:

For the production of an expression vector with a lower copy number compared to pBudBkt-tet, firstly the plasmid pBudBkt-tet(rev) was cleaved with Hind III and the 2 kb sized expression cassette for the acetolactate synthase gene isolated. pBudBkt-tet(rev) was isolated in the cloning of the vector pBudBkt-tet and contained the tetracycline expression cassette in opposite orientation to the acetolactate synthase expression cassette (FIG. 6). Next, the DNA fragment was cloned into the vector pACYC-LH cleaved with Hind III. The 5.3 kb expression vector pAC-BudBkt (FIG. 7) was produced. pACYC-LH is a 3.2 kb derivative of pACYC184 (FIG. 8), from which the chloramphenicol resistance marker had been removed. For this, the 4.2 kb vector pACYC184 was cleaved with Sca I and Bst 1107I and then religated.

### EXAMPLE 2

### Expression Analysis in E. coli

Plasmid DNA from the expression vectors pBudBkt-tet, pALSbl-tet and pAC-BudBkt was transformed by methods known per se into the E. coli strain JM105. As a control, E. coli JM105 transformed with the vector pACYC-LH was used. In each case, one clone was selected and cultivated in a shake flask culture. A pre-culture was produced from the E. coli strains in LBtet medium (10 g/l tryptone, 5 g/l yeast extract, 5 g/l NaCl, 15 μg/ml tetracycline) (culturing at 37° C. and 120 rpm overnight). 2 ml each of preculture were used as the inoculum of a main culture of 100 ml LBtet medium (300 ml conical flasks). The main cultures were shaken at 30° C. and 180 rpm until a cell density OD600 of 2.0 was reached. Then the inducer IPTG (isopropyl-B-thiogalactoside, 0.4 mM final concentration) was added and the mixture shaken overnight at 30° C. and 180 rpm.

For the analysis of the acetolactate synthase expression, 50 ml of the E. coli cells were centrifuged (10 mins 15,000 rpm, Sorvall centrifuge RC5C, fitted with an SS34 rotor), the cell pellet taken up in 2 ml KPi buffer (0.1 M potassium phosphate, 0.1 M NaCl, pH 7.0) disintegrated in a manner known per se with a so-called “French®Press” high pressure homogenizer (SLM-AMINCO) and a cell extract isolated by centrifugation (15 mins 15,000 rpm, Sorvall SS34 rotor). Aliquots of the cell extracts were used for the spectrophotometric determination of the acetolactate synthase activity. While in the control strain no activity could be measured, the specific acetolactate synthase activity in cell extracts was between 5 and 27 U/mg protein, depending on the particular construct.

Spectrophotometric determination of the acetolactate synthase activity:

The determination of the acetolactate synthase activity was performed in a manner known per se (Bauerle et al., Biochim. Biophys. Acta (1964) 92: 142-149). In this case, 1 U acetolactate synthase activity is defined as the quantity of enzyme which produces 1 μmol acetolactate/min under test conditions.

A suitable quantity of enzyme extract was adjusted to a volume of 2.5 ml together with test buffer (100 mM KPi, 10 mM MgCl2, pH 7.5), mixed with 2.5 ml 80 mM Na pyruvate and 160 μg/ml thiamine pyrophosphate, dissolved in H2O, and incubated at 37° C. After 0′, 10′, 30′ and 60′ incubation time, 1 ml of the mixture each time was pipetted into 0.1 ml of 50% H₂SO₄ and incubated for 30 mins at 37° C. in order to stop the reaction and to decarboxylate acetolactate formed to acetoin. Next, 0.9 ml of 2.5 M NaOH were added and 1.2 ml of the mixture was mixed with 0.2 ml of 0.5% creatine and 0.2 ml of 5% alpha-naphthol in 2.5 M NaOH and incubated for 60 mins at room temperature. Finally, the extinction at 524 nm was determined. Here the zero correction of the photometer was effected with a test mixture with buffer only, without enzyme.

The quantity of the acetolactate formed was determined from a standard curve previously created with acetoin.

For the determination of the specific activity, the protein concentration of the cell extracts was determined in a manner known per se with the so-called “BioRad Proteinassay” from BioRad.

### EXAMPLE 3

### Expression of Acetolactate Synthase in Klebsiella terrigena

The original strain was Klebsiella terrigena DSM 2687. The transformation with the plasmids pBudBkt-tet, pAC-BudBkt and pALSbl-tet was effected in a manner known per se analogously to the methods for transformation of E. coli familiar to those skilled in the art. As the control strain, the non-transformed wild type strain Klebsiella terrigena DSM 2687 was used.

Transformants were isolated and tested for acetolactate synthase activity by shake flask culturing. For this, in each case 50 ml FM2tet medium (without tetracycline in the case of the K. terrigena wild type control strain) was inoculated with a transformant and incubated for 24 hrs at 30° C. and 140 rpm (Infors shaker).

FM2tet medium contained glucose 60 g/l; 10 g/l; yeast extract (Oxoid) 2.5 g/l; ammonium sulfate 5 g/l; NaCl 0.5 g/l; FeSO₄×7H₂O 75 mg/l; Na₃ citrate×2 H₂O 1 g/l; CaCl₂×2H₂O 14.7 mg/l; MgSO₄×7H₂O 0.3 g/l; KH₂PO₄ 1.5 g/l; trace element mix 10 ml/l and tetracycline 15 mg/l. The pH of the FM2tet medium was adjusted to 6.0 before the start of culturing.

The trace element mix had the composition H₃BO₃ 2.5 g/l; CoCl₂×6H₂O 0.7 g/l; CuSO₄×5 H₂O 0.25 g/l; MnCl₂×4 H₂O 1.6 g/l; ZnSO₄×7H₂O 0.3 g/l and Na₂MoO₄×2H₂O 0.15 g/l.

The cells were analyzed as described in Example 2 for E. coli. Klebsiella cells were disintegrated with the “French®Press” and the cell extracts tested for acetolactate synthase activity. The specific acetolactate synthase activity in crude extracts of the various strains (determined as described in Example 2) is listed in Table 1.

TABLE 1
Comparison of the acetolactate synthase activity in
recombinant K. terrigena strains

| | | Acetolactate synthase | Relative |
|---|---|---|---|
| Strain | Construct | (U/mg) | activity |
| K. terrigena | — | 0.016 | 1 |
| WT | | | |
| K. terrigena | pALSbl-tet | 0.051 | 3.2 |
| pALSbl | | | |
| K. terrigena | pAC-BudBkt | 0.118 | 7.4 |
| pAC-BudBkt | | | |
| K. terrigena | pBudBkt-tet | 1.094 | 68.4 |
| pBudBkt-tet | | | |

### EXAMPLE 4

### 2,3-BDL Production by Shake Flask Culture of Recombinant K. terrigena Strains

The culturing of the K. terrigena strains transformed with the plasmids pBudBkt-tet, pAC-BudBkt and pALSbl-tet was effected as described in example 3. However, the culturing time was 96 hrs. In this case, the glucose concentration was determined at intervals of 24 hrs (glucose analyzer 7100MBS from YSI) and further glucose fed in as needed from a 40% (w/v) stock solution. At 48 hr intervals, samples were tested for their 2,3-BDL content. The 2,3-BDL production result is shown in Table 2.

TABLE 2
BDL production in Klebsiella terrigena transformants

| | K. terrigena | K. t.- | K. t.-pAC- | K. t.- |
|---|---|---|---|---|
| | WT | pBudBkt-tet | BudBkt | pALSbl-tet |
| Time | 2,3 BDL | 2,3 BDL | 2,3 BDL | 2,3 BDL |
| (hrs) | (g/l) | (g/l) | (g/l) | (g/l) |
| 48 | 23.2 | 32.5 | 30.7 | 32.8 |
| 96 | 34.7 | 49.3 | 48.1 | 48.7 |

As can be seen in Table 2, the overexpression of the acetolactate synthase genes from K. terrigena and B. licheniformis respectively in Klebsiella terrigena resulted in an unexpectedly high increase by ca. 40% in 2,3-BDL production in the shake flask culture. The production increase was independent of whether the acetolactate synthase activity of the recombinant strains had been increased by the factor 3 or else by the factor 68 (concerning this, compare example 3). Apparently a comparatively only slight overexpression suffices to obtain a significant increase of the 2,3-BDL production in shake flasks.

The determination of the 2,3-butanediol content in culture supernatants was effected in a manner known per se by ¹H-NMR. For this, an aliquot of the culture was centrifuged (10 mins 5000 rpm, Eppendorf Labofuge) and 0.1 ml of the culture supernatant was mixed with 0.6 ml TSP (3-(trimethylsilyl)propionic acid-2,2,3,3-d₄ sodium salt) standard solution of defined content (internal standard, typically 5 g/l) in D₂O. The ¹H-NMR analysis incl. peak integration was performed according to the prior art with an Avance 500 NMR instrument from Bruker. For the quantitative analysis, the NMR signals of the analytes were integrated in the following ranges:

TSP: 0.140-0.145 ppm (9H)

2,3-butanediol: 1.155-1.110 ppm (6H)

Ethanol: 1.205-1.157 ppm (3H)

Acetic acid: 2.000-1.965 ppm (3H)

Acetoin: 2.238-2.200 ppm (3H)

### EXAMPLE 5

2,3-BDL Production with Acetolactate Synthase-Overexpressing K. terrigena Strains by Fed Batch Fermentation

“Labfors II” fermenters from Infors were used. The working volume was 1.5 l (3 l fermenter volume). The fermenters were equipped according to the prior art with electrodes for measurement of the pO₂ and the pH and with a foam probe, which as needed regulated the metering in of an antifoam solution. The values from the measurement probes were recorded via a computer programme and displayed graphically. The fermentation parameters stirrer rotation rate (rpm), aeration (supply of compressed air in vvm, volume of compressed air per volume of fermentation medium per minute), pO₂ (oxygen partial pressure, relative oxygen content calibrated to an initial value of 100%), pH and fermentation temperature were controlled and recorded via a computer programme supplied by the fermenter manufacturer. Feed medium (74% glucose, w/v) was metered in via a peristaltic pump in accordance with the glucose consumption. To control foaming, a plant-based alkoxylated fatty acid ester, commercially available under the name Struktol J673 from Schill & Seilacher (20-25% v/v diluted in water), was used.

Strains used in the fermentation were the Klebsiella terrigena wild type strain (control strain from example 3) and the acetolactate synthase overproducing strains Klebsiella terrigena-pBudBkt-tet, Klebsiella terrigena-pAC-BudBkt and Klebsiella terrigena-pALSbl-tet (see example 4). The batch fermentation medium was FM2tet medium (see example 3, medium without tetracycline for the K. terrigena wild type control strain).

1.35 l of the medium were inoculated with 150 ml preculture. The preculture of the strain to be fermented was produced by 24 hr shake flask culturing in batch fermentation medium. The fermentation conditions were: temperature 30° C., stirrer rotation rate 1000 rpm, aeration with 1 vvm, pH 6.0.

At regular intervals, samples were withdrawn from the fermenter for the analysis of the following parameters:

The cell density OD600 as a measure of the biomass formed was determined photometrically at 600 nm (BioRad Photometer SmartSpec™ 3000).

For the determination of the dry biomass, for each measurement point in a threefold determination 1 ml of fermentation mixture was centrifuged and the cell pellet washed with water and dried to constant weight at 80° C.

The glucose content was determined as described in Example 4. The 2,3-BDL content was determined by NMR as described in Example 4.

After the glucose placed beforehand in the batch medium had been consumed, a 74% (w/v) glucose solution was fed in via a pump (peristaltic pump 101 U/R from Watson Marlow). In this case, the feeding rate was determined from the current glucose consumption rate.

Table 3 shows the time-dependent 2,3-BDL formation in the K. terrigena control strain and in the acetolactate synthase overproducing, recombinant Klebsiella terrigena strains.

As already observed in the shake flask experiments (Example 4), the overexpression of the acetolactate synthase with the expression plasmids pAC-BudBkt and pALSbl-tet leads to a significant increase in the 2,3-butanediol production by 25-30%, based on the maximum yield at 72 hrs fermentation time.

In contrast, surprisingly, as a result of the strong overexpression of acetolactate synthase with the plasmid pBudBkt-tet an inhibition of 2,3-BDL production was observed. This means that the 2,3-BDL yield in the fermentation can be increased by increasing the acetolactate synthase activity. However, excessively high activity of the acetolactate synthase unexpectedly leads to a decrease in the 2,3-BDL yield. The course of the production processes is shown in Table 3.

TABLE 3
Production of 2,3-BDL in K. terrigena strains by fed batch
fermentation

| | | K. t.- | | |
|---|---|---|---|---|
| | K. terrigena | pBudBkt- | K. t.-pAC- | K. t.- |
| | WT | tet | BudBkt | pALSbl |
| Time | 2,3 BDL | 2,3 BDL | 2,3 BDL | 2,3 BDL |
| (hrs) | (g/l) | (g/l) | (g/l) | (g/l) |
| 23 | 23.8 | 3.5 | 66.9 | 77.3 |
| 27 | 39.3 | 5.6 | 72.9 | 84.4 |
| 31 | 49.9 | 9.8 | 77.9 | 99.7 |
| 47 | 71.5 | 23.5 | 100.1 | 115.9 |
| 51 | 74.7 | 28.9 | 117.9 | 117.0 |
| 55 | 79.9 | 29.5 | 119.3 | 122.2 |
| 72 | 98.9 | 31.4 | 123.9 | 127.6 |

### EXAMPLE 6

### 2,3-BDL Fermentation on the 330 l Scale

The strain Klebsiella terrigena-pALSbl-tet was fermented (see Examples 4 and 5).

Production of an inoculum for the prefermenter: an inoculum of Klebsiella terrigena-pALSbl-tet in LBtet medium (see Example 2) was produced by inoculating 2×100 ml LBtet medium, each in a 1 l conical flask, in each case with 0.25 ml of a glycerine culture (overnight culturing of the strain in LBtet medium, treated with glycerine in a final concentration of 20% v/v and stored at −20° C.). The culturing was effected for 7 hrs at 30° C. and 120 rpm on an Infors orbital shaker (cell density OD₆₀₀/ml of 0.5-2.5). 100 ml of the preculture were used for the inoculation of 8 l fermenter medium. Two prefermenters each with 8 l of fermenter medium were inoculated.

Prefermenter: The fermentation was performed in two Biostat® C-DCU 3 fermenters from Sartorius BBI Systems GmbH. The fermentation medium was FM2tet (see example 3). The fermentation was effected in so-called batch mode.

2×8 l FM2tet were each inoculated with 100 ml inoculum. The fermentation temperature was 30° C. The pH of the fermentation was 6.0 and was kept constant with the correction agents 25% NH₄OH, or 6 N H₃PO₄. The aeration was effected with compressed air at a constant flow rate of 1 vvm. The oxygen partial pressure pO2 was adjusted to 50% saturation. The regulation of the oxygen partial pressure was effected via the stirring speed (stirrer rotation rate 450-1,000 rpm). To control foaming, Struktol J673 (20-25% v/v in water) was used. After 18 hrs fermentation time (cell density OD₆₀₀/ml of 30-40), the two prefermenters were used as inoculum for the main fermenter.

Main fermenter: The fermentation was performed in a Biostat® D 500 fermenter (working volume 330 l, vessel volume 500 l) from Sartorius BBI Systems GmbH. The fermentation medium was FM2tet (example 3). The fermentation was effected in so-called fed batch mode.

180 l FM2tet were inoculated with 16 l inoculum. The fermentation temperature was 30° C. The pH of the fermentation was 6.0 and was kept constant with the correction agents 25% NH₄OH, or 6 N H₃PO₄. The aeration was effected with compressed air at a constant flow rate of 1 vvm (see Example 4, based on the initial volume). The oxygen partial pressure pO2 was adjusted to 50% saturation. The regulation of the oxygen partial pressure was effected via the stirring speed (stirrer rotation rate 200-500 rpm). To control foaming, Struktol J673 (20-25% v/v in water) was used. In the course of the fermentation, the glucose consumption was determined by off-line glucose measurement with a glucose analyzer from YSI (see Example 4). As soon as the glucose concentration of the fermentation mixtures was ca. 20 g/l (8-10 hrs after inoculation), the metering in of a 60% w/w glucose feed solution was started. The flow rate of the feed was selected such that during the production phase a glucose concentration of 10-20 g/l could be maintained. After completion of the fermentation the volume in the fermenter was 330 l.

The analysis of the fermentation parameters was effected as described in Example 5. The course of the production process is shown in Table 4.

TABLE 4
Production of 2,3-BDL by fed batch fermentation on the 330 l
scale

| | K. t.-pALSbl-tet |
|---|---|
| Time | 2,3 BDL |
| (hrs) | (g/l) |
| 7.5 | 12.8 |
| 24 | 65.6 |
| 27 | 68 |
| 31 | 71.1 |
| 48 | 86.3 |
| 51 | 96.9 |
| 55 | 101.1 |
| 72 | 124.1 |

1. A production strain for producing 2,3-butanediol producible from an original strain of the species Klebsiella (Raoultella) terrigena or Klebsiella (Raoultella) planticola having a biosafety level of S1, wherein the production strain has an acetolactate synthase activity 3 to 30 times higher than the acetolactate synthase activity of the original strain, which is achieved b overexpression of a homologous or heterologous acetolactate synthase gene in the original strain.

2. The production strain as claimed in claim 1, wherein the acetolactate synthase activity of the production strain is 4 to 20 times higher than the acetolactate synthase activity of the original strain.

3. (canceled)

4. (canceled)

5. The production strain as claimed claim 1, wherein acetolactate synthase gene is derived from a bacterium of the genus Klebsiella (Raoultella) or Bacillus.

6. The production strain as claimed in claim 1, wherein the production strain was produced from a non-genetically optimized original strain and produces an acetolactate synthase in recombinant form with the result that 2,3-butanediol production is increased compared to the non-genetically optimized original strain by at least 25%, wherein the 2,3-butanediol yield of the original strain is at least 90 g/l.

7. A method for producing 2,3-butanediol, wherein a production strain as claimed in claim 1 is cultured in a growth medium.

8. The method as claimed in claim 7, wherein a culturing is effected in a pH range from pH 5 to pH 8 and a temperature range from 20° C. to 40° C. and anaerobically or aerobically with an oxygen supply by introduction of compressed air or pure oxygen and there is a culturing time for 2,3-butanediol production of 10 hrs to 200 hrs.

9. The method as claimed in claim 7, wherein a culturing is effected in a fermentation volume greater than 300 l.

10. The production strain as claimed claim 2, wherein the acetolactate synthase gene is derived from a bacterium of the genus Klebsiella (Raoultella) or Bacillus.

11. The production strain as claimed in claim 10, wherein the production strain was produced from a non-genetically optimized original strain and produces an acetolactate synthase in recombinant form with the result that 2,3-butanediol production is increased compared to the non-genetically optimized original strain by at least 100%, wherein the 2,3-butanediol yield of the original strain is at least 90 g/l.

12. The production strain as claimed in claim 1, wherein the production strain was produced from a non-genetically optimized original strain and produces an acetolactate synthase in recombinant form with the result that 2,3-butanediol production is increased compared to the non-genetically optimized original strain by at least 100%, wherein the 2,3-butanediol yield of the original strain is at least 90 g/l.

13. The method as claimed in claim 8, wherein a culturing is effected in a fermentation volume greater than 300 l.
